SEQUENCE LISTING (1 sequence)

SEQ ID NO: 1 - protein, 461 aa, Artificial
  Fusion protein
MQTFTMVLEEIWTSLFMWFFYALIPCLLTDEVAILPAPQTLSVLSTNMKHLLMWSPVIAPGETVYYSVEYQGEYESLYTSHIWIPSSWCSLTEGPECDVTDDITATVPYNLRVRATLGSQTSAWSILKHPFNRNSTILTRPGMEITKDGFHLVIELEDLGPQFEFLVAYWRREPGAEEHVKMVRSGGIPVHLETMEPGAAYCVKAQTFVKAIGRYSAFSQTECVEVQGEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2018/0057564 A1
(43) Pub. Date: Mar. 1, 2018

(54) ANTAGONISTS OF IL-20 FAMILY OF CYTOKINES AND THEIR USE

(71) Applicant: Peng Liang, Nashville, TN (US)

(72) Inventor: Peng Liang, Nashville, TN (US)

(73) Assignee: Clover Biopharmaceuticals, Chengdu (CN)

(21) Appl. No.: 15/244,510

(22) Filed: Aug. 23, 2016

(51) Int. Cl. C07K 14/715; C07K 16/28

(52) U.S. Cl. CPC C07K 14/7155; C07K 16/2866; C07K 2319/30; C07K 2319/32; C07K 2317/76; C07K 2319/70; C07K 2317/24; C07K 2317/55; C07K 2317/31; C07K 2317/622; C07K 2317/21

(57) ABSTRACT

Methods for blocking the activity of the IL-20 family of cytokines in a mammal having a disorder-mediated by one or more of the IL-20 family of cytokines consisting of IL-19, IL-20 and IL-24 are disclosed. The methods involve administering a therapeutically effective amount of either a soluble IL-20R2-IgG Fc fusion protein or a neutralizing monoclonal antibody to IL-20R2, both of which are able to block the biological functions of all three members of IL-20 family of cytokines.

### FIELD OF THE INVENTION

This invention relates to recombinant soluble receptor-antibody fusion proteins and antagonist monoclonal antibodies to such receptor and their uses for simultaneously blocking the biological and pathological functions of the corresponding multiple ligands. Such receptor-antibody fusion proteins and antagonist monoclonal antibodies may be used for the treatment of autoimmune diseases and cancer.

### BACKGROUND OF THE INVENTION

IL-19, IL-20 and IL-24 are members of IL-20 family cytokines. These three cytokines share a common β receptor subunit IL-20R2 with all three being able to signal through IL-20R1/IL-20R2, while IL-20 and IL-24 can also signal through IL-22R1/IL-20R2 heterodimeric receptor complex. Such significant receptor sharing among these cytokines has raised questions about whether or not the three cytokines have redundant biological functions in vivo or if they may use the same receptors for different biological endpoints in a tissue-specific or temporally regulated manner. Substantial evidence supports the linkage between the development of autoimmune diseases—such as rheumatoid arthritis (RA), psoriasis, osteoporosis and atopic dermatitis, as well as cancer—and the abnormal overexpression of IL-19, IL-20 and IL-24, with synovial tissues having been identified as a major source of the three cytokines in patients with RA.

A neutralizing antibody against IL-20 was shown to be able to attenuate disease severity of collagen-induced arthritis (CIA) in rats. Similar efficacy was also reported for anti-IL-19 mAb in the same animal model, with IL-24-specific blockers yet to be tested. Although in these studies, both anti-IL-20 and anti-IL-19 mAbs exhibited similar efficacy to TNF blockers such as etanercept in reducing disease symptoms in a rat CIA model, combination therapy for simultaneously blocking IL-20 and anti-IL-19 signaling has yet to be studied and would be expected to result in a better outcome. In contrast to complete failure for treating psoriasis in human clinical trials, anti-IL-20 mAb has demonstrated some moderate therapeutic effect against RA in a Phase 2 clinical trial, although seen as inferior to anti-TNF biologics. Previous transgenic studies showed that IL-24 had overlapping functions with IL-20 in the epidermis. Partially purified and characterized recombinant soluble IL-20R2-Fc fusion proteins were shown to have variable specificity and potency towards its ligands and failed to bind to IL-20 in cell based assays. Given the promiscuous nature of receptor sharing for the IL-20 sub-family of cytokines, it would be clearly advantageous to develop biologic therapies that could simultaneously block signaling of all three cytokines that signal through receptor complexes involving IL-20R2.

### SUMMARY OF THE INVENTION

Two heterodimeric receptors consisting of either IL-20R1 or IL-22R1 in complex with a common β receptor subunit IL-20R2 are shared by three of the IL-20 family of cytokines, IL-19, IL-20 and IL-24. These pro-inflammatory cytokines have been implicated in the pathogenesis of a number of autoimmune diseases, including rheumatoid arthritis (RA), psoriasis and atopic dermatitis, as well as in cancer.

Disclosed here is a discovery of a human soluble IL-20R2-Fc fusion protein (rhIL-20R2-Fc) expressed by in-frame fusing a DNA template encoding the soluble receptor of human IL-20R2 with that of human Ig G1 Fc region is being able to directly bind to all three family members from IL-20 family, namely, IL-19, IL-20 and IL-24 with similar high affinity, and potently inhibits the biological functions of these individual ligands in Ba/F3 cell-based receptor signaling in vitro, as well as blocks collagen induced arthritis (CIA) in DBA/1 mouse model in vivo. In DBA/1 mouse CIA model, rhIL-20R2-Fc exhibits comparable efficacy as TNF blocker etanercept in the treatment of established arthritis, while the combined use of both biologics manifests little synergistic therapeutic effects. In situ ligand-receptor functional binding analysis shows that a large amount of immune infiltrates expressing high levels of TNFR and IL-20 family of cytokines congregate within the inflamed disease tissues. Co-localization experiments reveal that signals from IL-20R2 and TNF transduction pathways seem to converge in macrophages and function in tandem in orchestrating the pathogenesis of RA. Elucidation of this interaction provides a better understanding of cytokine cross-talk in RA and potentially in other autoimmune diseases as well as in cancer, and a rationale for more effective biologic therapies that target IL-20R2, instead of individual cytokines from IL-20 family.

This important discovery also provides a strong evidence for IL-20R2 receptor subunit being directly involved in binding to all three ligands from IL-20 family. From this finding, it is also predicted that certain antibodies raised against IL-20R2 may pertain broadly neutralizing activity against all three ligands from the IL-20 family by binding to the ligand contact regions of the IL-20R2. Such broadly neutralizing IL-20R2 antibodies and IL-20R2-Fc may be used as therapeutic antagonists against diseases caused by the abnormal expression of IL-20 family of cytokines.

In one aspect of the invention, a method for blocking the activity of the IL-20 family of cytokines in a mammal having a disorder-mediated by one or more of the IL-20 family of cytokines is disclosed, which comprises administering to said mammal via either subcutaneous or intravenous injection of a therapeutically effective amount of a soluble IL-20R2-IgG Fc fusion protein.

Within one embodiment, the disorder-mediated by one or more of the IL-20 family of cytokines is an inflammatory disorders which includes psoriasis, atopic dermatitis, Cohn's disease, heart disease, and wound healing disorder.

In a preferred embodiment, the disorder-mediated by one or more of the IL-20 family of cytokines is an arthritis condition. In another preferred embodiment, the disorder-mediated by one or more of the IL-20 family of cytokines is cancer.

Within one embodiment, the soluble IL-20R2-IgG Fc fusion protein is administered by intravenous injection. In another embodiment, the soluble IL-20R2-IgG Fc fusion protein is administered by subcutaneous injection. In yet another embodiment, the soluble IL-20R2-IgG Fc fusion protein is administered by a series of doses separated by intervals of days or weeks.

Within another embodiment, the soluble IL-20R2 in IL-20R2-IgG Fc fusion protein is a human receptor subunit. In another embodiment, the IgG Fc in IL-20R2-IgG Fc fusion protein is from human IgG1.

In a preferred embodiment, the soluble IL-20R2-IgG Fc fusion protein is identified by SEQ ID. No. 1.

Within yet another embodiment, the IgG Fc in IL-20R2-IgG Fc fusion protein is selected from a group consisting of human IgG1, IgG2, IgG3, IgG4 and IgA.

In another aspect of the invention, a method is disclosed for blocking the activity of the IL-20 family of cytokines in a mammal having an disorder-mediated by one or more of the IL-20 family of cytokines which comprises administering to said mammal a therapeutically effective amount of a neutralizing monoclonal antibody against IL-20R2, wherein the neutralizing monoclonal antibody upon binding to IL-20R2 receptor subunit within its functional heterodimeric receptor complexes on a target cell surface, prevents all three cytokines from IL-20 family (IL-19, IL-20 and IL-24) from binding to and signaling from these receptors.

In one aspect of the invention, a method for blocking the activity of the IL-20 family of cytokines in a mammal having a disorder-mediated by one or more of the IL-20 family of cytokines is disclosed, which comprises administering to said mammal via either subcutaneous or intravenous injection of a therapeutically effective amount of a neutralizing monoclonal antibody against IL-20R2.

Within one embodiment, the disorder-mediated by one or more of the IL-20 family of cytokines is an inflammatory disorders which includes psoriasis, atopic dermatitis, Cohn's disease, heart disease, and wound healing disorder.

In a preferred embodiment, the disorder-mediated by one or more of the IL-20 family of cytokines is an arthritis condition. In another preferred embodiment, the disorder-mediated by one or more of the IL-20 family of cytokines is cancer.

Within one embodiment, the neutralizing monoclonal antibody against IL-20R2 is administered by intravenous injection. In another embodiment, the neutralizing monoclonal antibody against IL-20R2 is administered by subcutaneous injection. In yet another embodiment, the neutralizing monoclonal antibody against the IL-20R2 is administered by a series of doses separated by intervals of days or weeks.

Within another embodiment, the neutralizing monoclonal antibody is against human IL-20R2 receptor subunit.

Within embodiment, the neutralizing monoclonal antibody against the IL-20R2 is a humanized antibody. Within another embodiment, the neutralizing monoclonal antibody against the IL-20R2 is chimeric antibody. Within yet another embodiment, the neutralizing monoclonal antibody against the IL-20R2 is a Fab or part of part of a bi-specific antibody.

In a preferred embodiment, the neutralizing monoclonal antibody against IL-20R2 is a fully human antibody. In yet another preferred embodiment, the neutralizing monoclonal antibody against IL-20R2 is a fully human antibody selected from IgG1, IgG2, IgG3, IgG4 or IgA subtype.

### BRIEF DESCRIPTION OF THE DRAWINGS

FIG. 1A provides a view of high-level expression and affinity purification of rhIL-20R2-Fc fusion protein. Specifically, a 10% SDS-PAGE analysis of rhIL-20R2-Fc expression from a fed-batch serum-free cell culture in the bioreactor is shown. 10 μL of cell-free conditioned medium from Day 9 to Day 16 were analyzed under non-reducing condition followed by Coomassie Blue staining.

FIG. 1B provides a view of high-level expression and affinity purification of rhIL-20R2-Fc fusion protein. Specifically, an ELISA analysis of rhIL-20R2-Fc production in conditioned medium from Day 9 to Day 16 with 1 mg/mL of rhIL-20R2-Fc standard as a control is shown.

FIG. 1C provides a view of high-level expression and affinity purification of rhIL-20R2-Fc fusion protein. Specifically, a SDS-PAGE analysis of purified rhIL-20R2-Fc (2 μg per lane) under either non-reducing or reducing conditions with Coomassie Blue staining is shown. Lane 1: molecular weight marker; Lane 2: rhIL-20R2-Fc without β-ME; Lane 3: rhIL-20R2-Fc with β-ME.

FIG. 1D provides a view of high-level expression and affinity purification of rhIL-20R2-Fc fusion protein. Specifically, a purity evaluation of rhIL-20R2-Fc by SEC-HPLC, with OD₂₈₀ detection. The main peak area of rhIL-20R2-Fc was 98.1%.

FIG. 2A Shows conditioned media from CHO cells or 293T cells expressing AP, IL-19-AP, IL-20-AP and IL-24-AP were analyzed by Western blot using anti-AP antibody to confirm the size and integrity of the ligand-AP fusions. rhIL-20R2-Fc binds to IL-19, IL-20 and IL-24 with similar affinity.

FIG. 2B shows ligand-receptor affinity staining analysis of rhIL-20R2-Fc binding to ligand-AP fusion proteins. 2 μg of rhTNFR2-Fc and rhIL-20R2-Fc were each separated by non-reducing SDS-PAGE and either visualized by Coomassie Blue staining or transferred to a PVDF membrane followed by in situ detection with corresponding AP labeled ligands. rhIL-20R2-Fc but not rhTNFR2-Fc was detected by human IL-19-AP, IL-20-AP and IL-24-AP, but not by AP alone. rhTNFR2-Fc which was detected by its ligand-AP (TNFα-AP) served as a control for ligand specificity for the analysis.

FIG. 2C provides ELISA assays for native rhIL-20R2-Fc binding by equal concentration of AP labeled cytokines from IL-20 sub-family, with AP alone as a negative control. The results were representative of three independent experiments.

FIG. 2D provides a chart showing quantitative binding kinetics study of AP tagged ligands to rhIL-20R2-Fc captured by protein A were determined by ELISA with increasing concentration of corresponding AP labeled ligands. Each data point was from average of triplicated measurements. The ligand-mediated receptor binding was derived after subtracting the nonspecific binding (AP alone) and analyzed by kinetic program “HYPER”. The dissociation constants (K_{d}) for all three cytokines from IL-20 sub-family were about 8-10 nM.

FIG. 2E provides a blockade of ligand-dependent cell proliferation with rhIL-20R2-Fc. Ba/F3 (IL-22R1/IL-20R2) cells were cultured with either 1 U/mL (1 μg/mL) of IL-19-AP, IL-20-AP or IL-24-AP in the presence or absence of 100 μg/mL of rhIL-20R2-Fc. Mouse IL-3 at 1 ng/mL was used as a control for the target specificity for rhIL-20R2-Fc.

FIG. 3A shows acute expression of IL-20 family cytokines and TNF receptor (TNFR) from immune infiltrates in RA. Specifically, paraffin embedded sections (original magnification×10) from CIA model mice and normal control were analyzed by in situ ligand or receptor affinity staining with IL-20R2-AP, TNFα-AP and AP alone, respectively, to detect cell types expressing IL-20R2 sub-family of cytokines and TNFα receptor (TNFR). Large number of immune infiltrates over expressing both IL-20R2 sub-family of cytokines and TNFR were seen (blue color signals) in the disease tissues but not in normal controls. The results were representative of multiple independent experiments.

FIG. 3B shows the specificity of IL-20R2-AP and TNFα-AP ligand-receptor affinity staining was confirmed by competing with 10-fold excess of rhIL-20R2-Fc and TNFα, which essentially completely blocked the ligand-AP and receptor-AP binding. The same concentration of irrelevant competitor protein served as negative controls for the specificity of the competition experiments.

FIG. 3C shows colocalization of IL-20R2 sub-family of cytokines and TNFR expression in the same immune infiltrates by sequential double staining with IL-20R2-biotin followed by TNFα-AP on a same CIA disease tissue section (magnification ×20). IL-20R2-biotin binding signals were in light brown color, while overlapping signals from double staining yield dark brown color from the immune infiltrates (Blue over light brown color).

FIG. 3D provides a view of cell type identification for the immune infiltrates over-expressing IL-20 family cytokines and TNFR in DBA/1 mouse CIA model. A same CIA diseased tissue slide was sequentially double stained with anti-F4/80 (macrophage specific) followed by TNFα-AP (TNFR specific) and the signals from each were sequentially captured by an upright microscope (original magnification 20×). Immune infiltrates that stained positive for anti-F4/80 (in light brown color) overlapped with the signal from TNFα-AP staining (purple color) (AP staining color in blue over light brown color from HRP).

FIG. 4A provides pharmacokinetics analysis of rhIL-20R2-Fc. Specifically, plasma concentrations versus time profile of rhIL-20R2-Fc after intraperitoneal (IP) administration of rhIL-20R2-Fc in DBA/1 mouse.

FIG. 4B shows plasma concentration (log scale) versus time profile of rhIL-20R2-Fc after IP administration in DBA/1 mouse. Data represents mean±SD (n=3).

FIG. 5A demonstrates inhibition of collagen induced arthritis by rhIL-20R2-Fc. Specifically, the figure shows comparison of arthritis scores from DBA/1 mice treated with either rhIL-20R2-Fc or rhTNFR2-Fc (etanercept) alone, and in drug combination. The results were representative of four independent observers.

FIG. 5B shows representative photographs of the hind paws of CIA mice on Day 21. As compared with healthy control and treatment groups, the hind paws from vehicle group of mice were significantly swollen.

FIG. 5C shows representative images of infrared thermography (IRT) of the hind paws of the CIA mice on Day 21. In comparison with healthy control and treatment groups, hind paws from vehicle group of mice showed significantly higher temperature and severe swelling (indicated by arrows).

FIG. 5D shows a comparison of hind paws' temperature from DBA/1 mice treated with either rhIL-20R2-Fc or rhTNFR2-Fc (etanercept) alone, and in drug combination. Regional temperature was analyzed by XJ-Infrared report analysis software. Values are mean±SD (n=6).

FIG. 5E provides representative images of radiograph of the hind paws of CIA mice on Day 21. As compared with healthy control and treatment groups, hind paws from vehicle group of mice were significantly deformed, swollen and with joint space narrowing (indicated by arrows).

FIG. 5F provides a comparison of the degree of joint swelling and bone erosion from DBA/1 mice treated with either rhIL-20R2-Fc or rhTNFR2-Fc (etanercept) alone, and in drug combination. Values are mean±SD (n=6). The results were from a representative experiment.

FIG. 6A shows detection of abnormal overexpression of both the ligands and receptors from the IL-20 sub-family of cytokines in human colorectal cancer. Alkaline phosphatase-tagged (AP-Tagged) ligand and soluble IL-20R2 were used as probes to conduct in situ staining of paraffin-embedded human colorectal cancer tissue specimens and visualized by AP substrate that produced purple color. Cancer epithelial cells, but not the adjacent normal tissues or stromal cells from multiple human colorectal cancer patients, were stained strikingly positive for both the ligand and receptor expression using either 1 Unit/mL of human IL-24-AP or human soluble IL-20R2-AP as probe.

FIG. 6B shows further detection of abnormal overexpression of both the ligands and receptors from the IL-20 sub-family of cytokines in human colorectal cancer. Alkaline phosphatase-tagged (AP-Tagged) ligand and soluble IL-20R2 were used as probes to conduct in situ staining of paraffin-embedded human colorectal cancer tissue specimens and visualized by AP substrate that produced purple color. Cancer epithelial cells, but not the adjacent normal tissues or stromal cells from multiple human colorectal cancer patients, were stained strikingly positive for both the ligand and receptor expression using either 1 Unit/mL of human IL-24-AP or human soluble IL-20R2-AP as probe.

### DESCRIPTION OF SEQUENCE LISTING

SEQ ID NO. 1 Shows the amino acid sequence of recombinant human IL-20R2 and IgG1 Fc fusion protein (rhIL-20R2-Fc).

### DETAILED DESCRIPTION OF THE INVENTION

The detailed description set forth below in connection with the appended drawings and sequence listing is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Prior to setting forth the invention, it may be helpful to an understanding thereof to set forth definitions of certain terms to be used hereinafter.

DNA Construct: A DNA molecule, generally in the form of a plasmid or viral vector, either single- or double-stranded that has been modified through recombinant DNA technology to contain segments of DNA joined in a manner that as a whole would not otherwise exist in nature. DNA constructs contain the information necessary to direct the expression and/or secretion of the encoding protein of interest.

Signal Peptide Sequence: A stretch of amino acid sequence that acts to direct the secretion of a mature polypeptide or protein from a cell. Signal peptides are characterized by a core of hydrophobic amino acids and are typically found at the amino termini of newly synthesized proteins to be secreted or anchored on the cell surface. The signal peptide is often cleaved from the mature protein during secretion. Such signal peptides contain processing sites that allow cleavage of the signal peptides from the mature proteins as it passes through the protein secretory pathway. A signal peptide sequence when linked to the amino terminus of another protein without a signal peptide can direct the secretion of the fused protein. Most of the secreted proteins, such as growth factors, peptide hormones, cytokines and membrane proteins, such as cell surface receptors, contain a signal peptide sequence when synthesized as a nascent protein.

Soluble receptor: The extracellular domain, in part or as a whole, of a cell surface receptor, which is capable of binding its ligand. Generally, it does not contain any internal stretch of hydrophobic amino acid sequence responsible for membrane anchoring.

Fc fusion proteins: Fc-fusion proteins are recombinant polypeptides that join the constant region (Fc domain) of an antibody with another biologically active protein, such as a soluble receptor, to generate a molecule with unique structure-function properties and significant therapeutic potential. Soluble receptor-Fc fusion proteins form dimeric conformation and function much like antibodies in binding to their target ligands, thereby inhibiting their biological functions. Many of such proteins have been approved for clinical use, including Enbrel which is the soluble human TNFRII fused with human Ig G1 Fc to treat a number of auto-immune diseases caused by abnormal expression of TNF-alpha, and Elyea which is a soluble VEGFR fusion with human Ig G1 Fc to treat Age-Related Macular Degeneration (AMD) due to increased VEGF expression in the eyes.

Monoclonal antibodies: Monoclonal antibodies (mAb) are antibodies that are made by identical immune cells that come from a unique parent B cell clone, in contrast to polyclonal antibodies, which are made from multiple different immune cells. Monoclonal antibodies can have monovalent affinity, in that they bind to the same epitope (the part of an antigen that is recognized by the antibody), thus have a defined target specificity and biological functions.

Neutralizing monoclonal antibody: a neutralizing monoclonal antibody refers to a monoclonal antibody that not only binds to its target antigen, but also inhibits its biological functions.

IL-20 family cytokines: IL-20 family cytokines consists of IL-19, IL-20 and IL-24. These three cytokines share a common β receptor subunit IL-20R2 with all three being able to signal through IL-20R1/IL-20R2 heterodimeric receptor complex, while IL-20 and IL-24 can also signal through IL-22R1/IL-20R2 heterodimeric receptor complex. Such extensive receptor sharing among these cytokines implies that the three cytokines from the IL-20 family may have redundant biological functions in vivo. Although IL-20R2 does not seem to be directly involved in signal transduction due to its short cytoplasmic domain, in contrast to that of IL-20R1 and IL-22R1, it was shown to be directly involved in IL-24 binding.

Prior to the current invention, no information about whether IL-20R2 can bind to IL-19 and IL-20 is available; nor were any antibodies or soluble receptors known to bind to and block the biological functions of all three members of IL-20 family of cytokines.

The present invention, in both fusion protein and monoclonal antibody embodiments, is not naturally occurring, and both embodiments have markedly different characteristics from any allegedly naturally occurring counterparts (of which there are none). Namely, all embodiments of the present invention have different structural characteristics when compared to any naturally occurring matter. These structural differences lead to different functional characteristics, and no mutations like those claimed are known or present in nature. Thus, all embodiments of the present invention are markedly different from any natural matter because the present invention has both structural and functional differences compared to anything found in nature.

High-Level Expression and Purification of rhIL-20R2-Fc Fusion Protein

To test if IL-20R2 can also bind to the other two ligands in the IL-20 family and be used as a broad antagonist for signaling through receptor complexes involving IL-20R2, we developed a CHO cell line and complementary serum free cell culture process from which rhIL-20R2-Fc can be produced at high level. To do so, cDNA encoding human IL-20R2 soluble receptor was in-frame fused to human IgG1 Fc in pGH-Fc expression vector (GenHunter Corporation) at HindIII and BgIII, and was stably transfected into GH-CHO (dhfr −/−) cell line (GenHunter Corporation) using FUGENE 6 (ROCHE) grown in IMDM medium with 10% FBS and 1% Penn-Strep supplemented with HT (Sigma). After stepwise gene amplification with increasing concentrations (0-1 μM) of MTX (Sigma), the clone with highest rhIL-20R2-Fc titer assayed by ELISA with protein A capture (5 μg/ml) and detection by IL-24-AP was obtained. The cells were then adapted to SFM-4-CHO (Hyclone) serum free medium and rhIL-20R2-Fc was produced in a 14 L Celligen bioreactor (Eppendorf) under Fed-batch process with CellBoost 5 (Hyclone) added every other day from Day 3 until harvest. As a result, large quantity of highly purified rhIL-20R2-Fc was obtained and tested for ligand binding.

Based on earlier findings that IL-20R2 alone, when expressed either on the surface of transfected Cos-1 cells or as soluble receptor-Fc fusion, could directly bind to IL-24, we tried to test if rhIL-20R2-Fc could also bind to IL-19 and IL-20, and thus may serve as a better biologic blocker against the IL-20R mediated signaling than any antibodies to individual cytokines previously attempted and failed in the clinic. Previous indirect binding assays based on the neutralization of either ligand-mediated signal transduction or receptor binding resulted in inconsistent findings. It seemed from these studies that rhIL-20R2-Fc could not bind to IL-20, while its ability to block IL-19 and IL-24 differed in potency depending on which assays were used.

### Example 1

rhIL-20R2-Fc Binds to all Three Cytokines in IL-20 Family In Vitro with Equal Affinity in Vitro

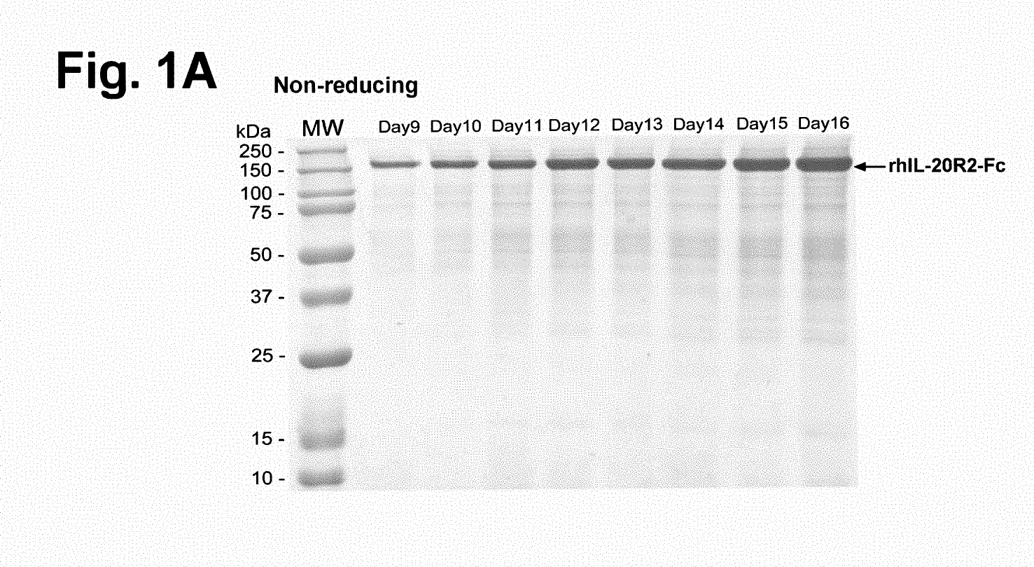

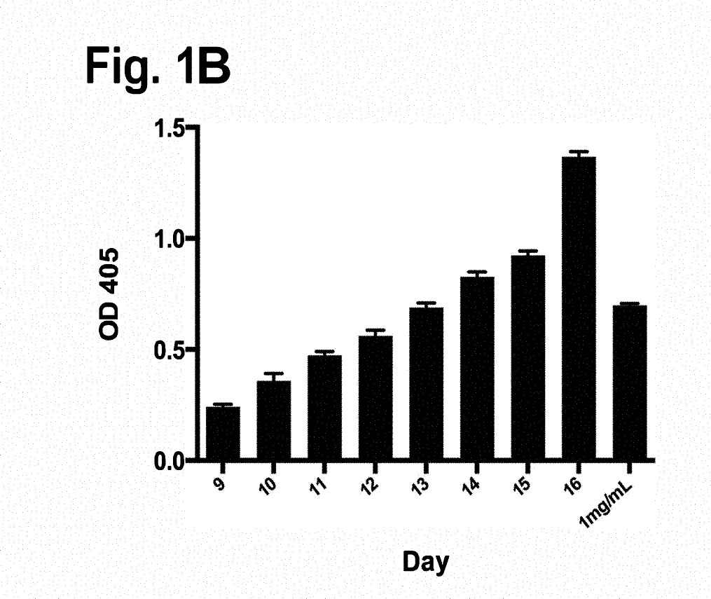

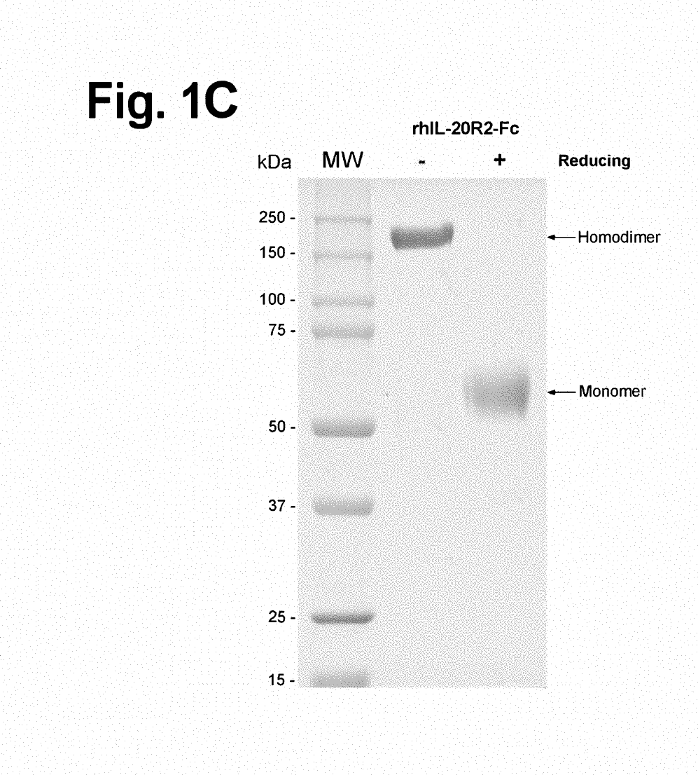

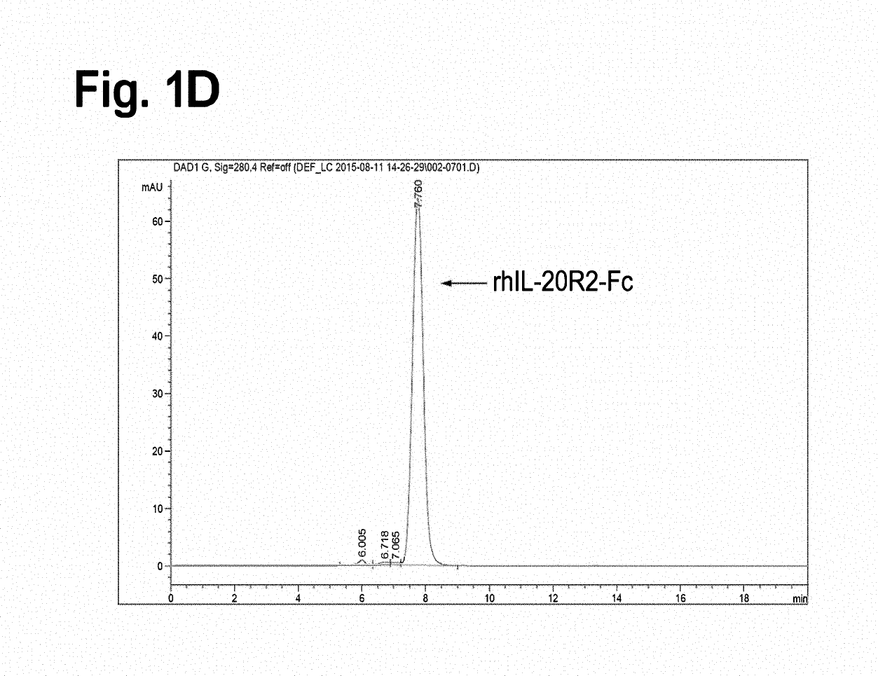

To better determine and explore the biochemical and therapeutic functions of rhIL-20R2-Fc, we screened for high-titer production clones of transfected CHO cells via MTX-mediated gene amplification. The resulting leading clone was adapted to serum free medium and fed-batch cell culture from bioreactor led to high-level expression of rhIL-20R2-Fc close to 2 g/L (FIGS. 1A and B). rhIL-20R2-Fc was purified to near homogeneity via protein A-based affinity chromatography followed by gel filtration (FIGS. 1C and D).

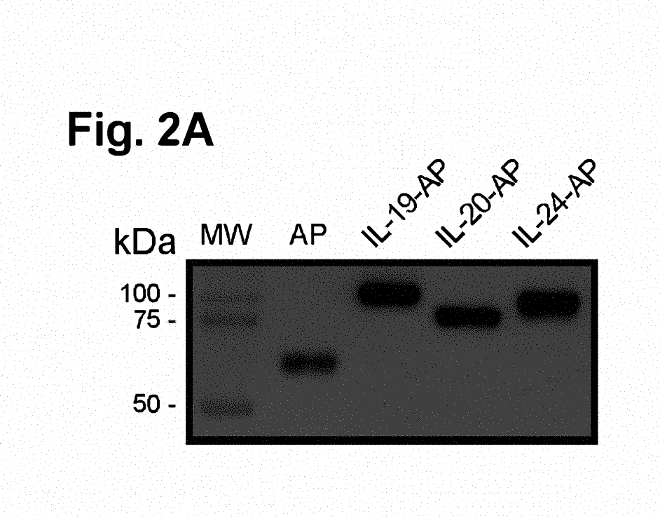

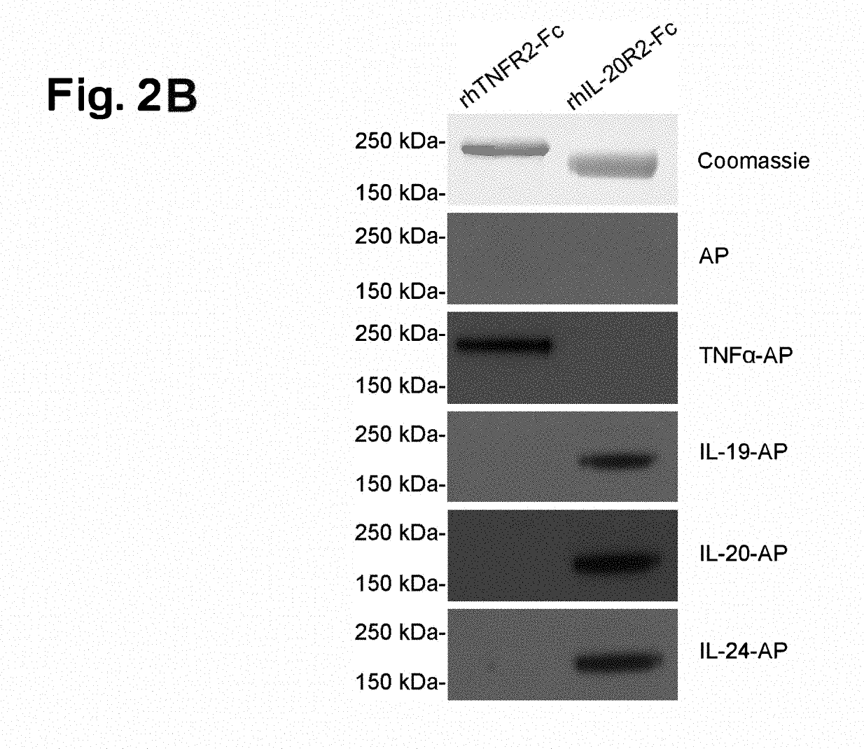

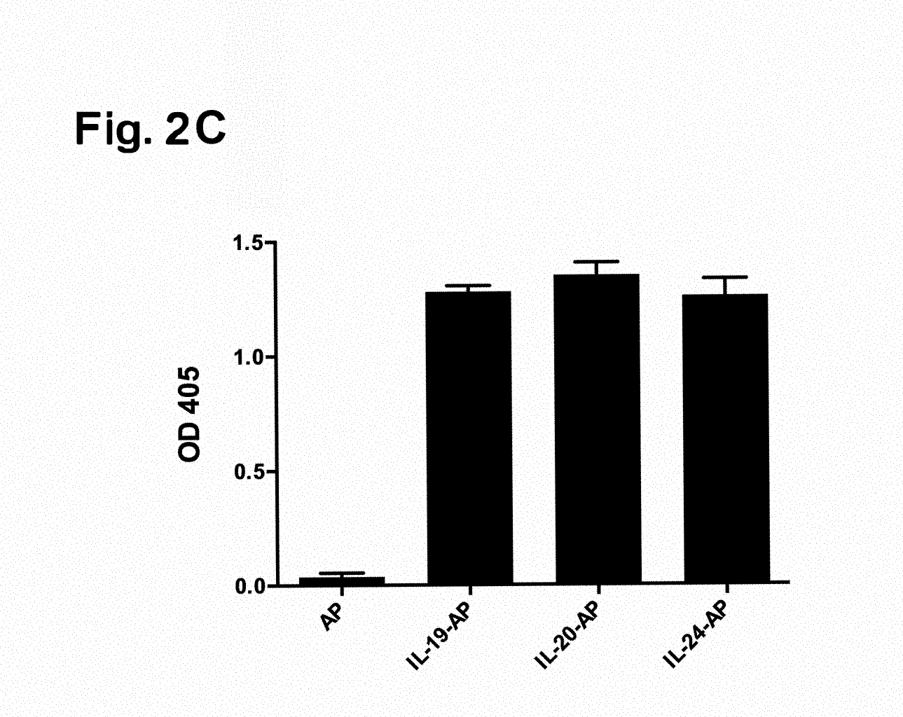

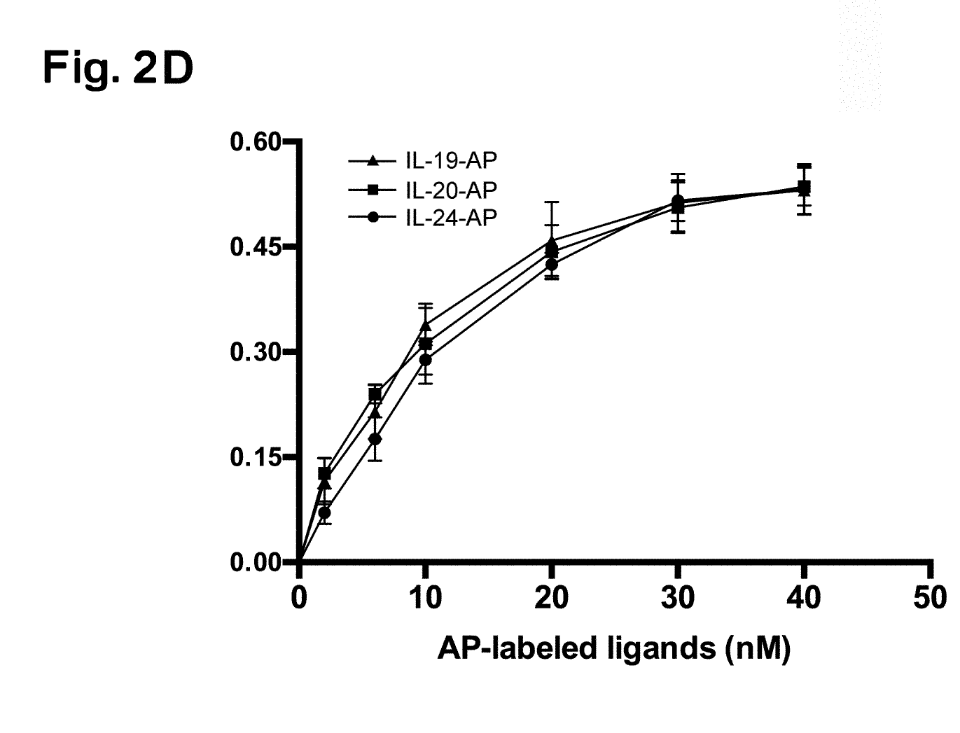

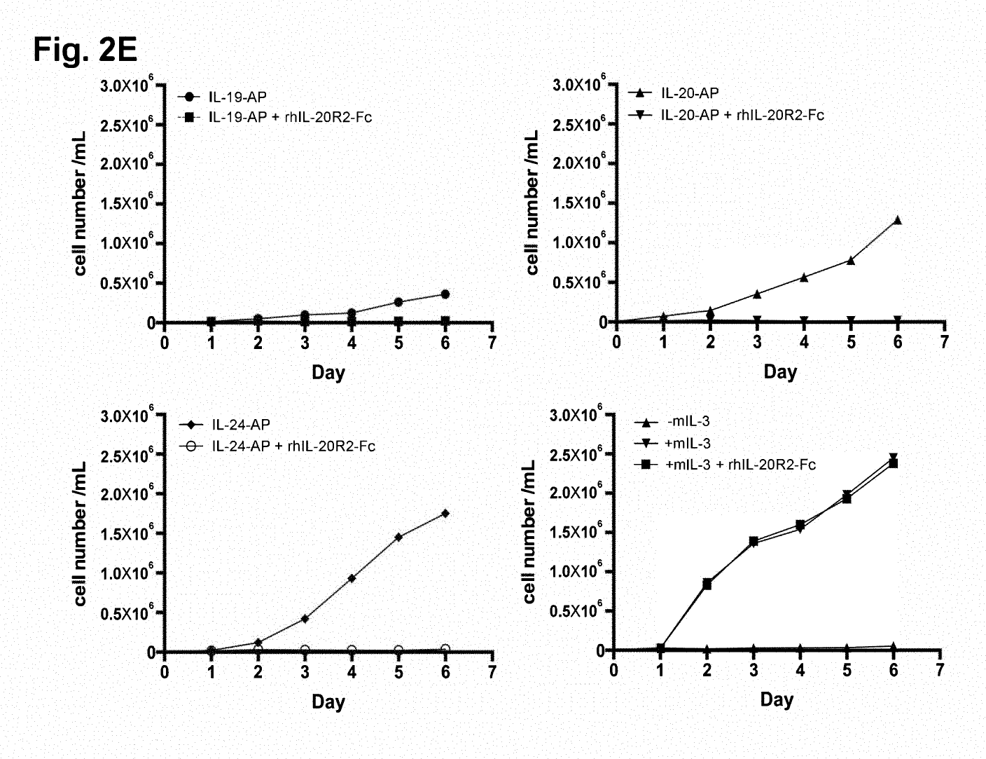

To determine whether the rhIL-20R2-Fc fusion protein could bind to the three ligands in IL-20 sub-family, we employed AP-tagged human IL-19, IL-20 and IL-24 (hIL-19-AP, hIL-20-AP, and hIL-24-AP) as probes to conduct in situ ligand-receptor affinity binding. After confirming the expression of hIL-19-AP, hIL-20-AP, hIL-24-AP and AP control as secreted proteins (FIG. 2A), these AP-Tag ligands were each tested for in situ receptor binding after SDS-PAGE separation and transfer of the purified rhIL-20R2-Fc fusion protein to a PVDF membrane. The result indicated that under non-reducing condition, the rhIL-20R2-Fc could bind to all three AP-tagged cytokines, while AP alone failed to do so (FIG. 2B). As a control, a recombinant human soluble TNFR2-Fc fusion protein was also included for the in situ binding assay, which confirmed the specificity of the ligand-receptor interactions. To more accurately determine the binding affinity of each AP-Tagged ligand to rhIL-20R2-Fc, we then conducted quantitative ELISA by first capturing the soluble receptor-Fc fusion protein via protein A coating. Then increasing concentration of each AP-Tagged ligand was allowed to saturate the receptor and the results indicated that hIL-19-AP, hIL-20-AP, hIL-24-AP all bound to rhIL-20R2-Fc with similar saturation kinetics with an estimated Kd about 10 nM (FIGS. 2C and D).

### Example 2

rhIL-20R2-Fc Blocks the Ligand-Dependent Cell Proliferation.

To further demonstrate that the rhIL-20R2-Fc could inhibit the cell signaling from each of the three IL-20 family of cytokines in vitro, we employed a Ba/F3 cell line stably expressing IL-22R1/IL-20R2. We showed that rhIL-20R2-Fc at 100 μg/mL (about 100× excess to ligands) could completely block both IL-20-AP and IL-24-AP mediated cell proliferation, while having little effect on IL-3 dependent growth (FIG. 2 E). Although it has been reported that IL-19 could not signal through IL-22R1/IL-20R2, we observed that IL-19-AP supported a residual cell survival and growth in this cell line, which was also completely abolished by rhIL-20R2-Fc. We believe that this important bioassay further establishes that rhIL-20R2-Fc can functionally block the receptor signaling via each individual cytokines from the IL-20 sub-family.

### Example 3

Detection of Acute Expression of IL-20 Family of Cytokines and TNF Receptor (TNFR) from Immune Infiltrates in Mouse CIA Model

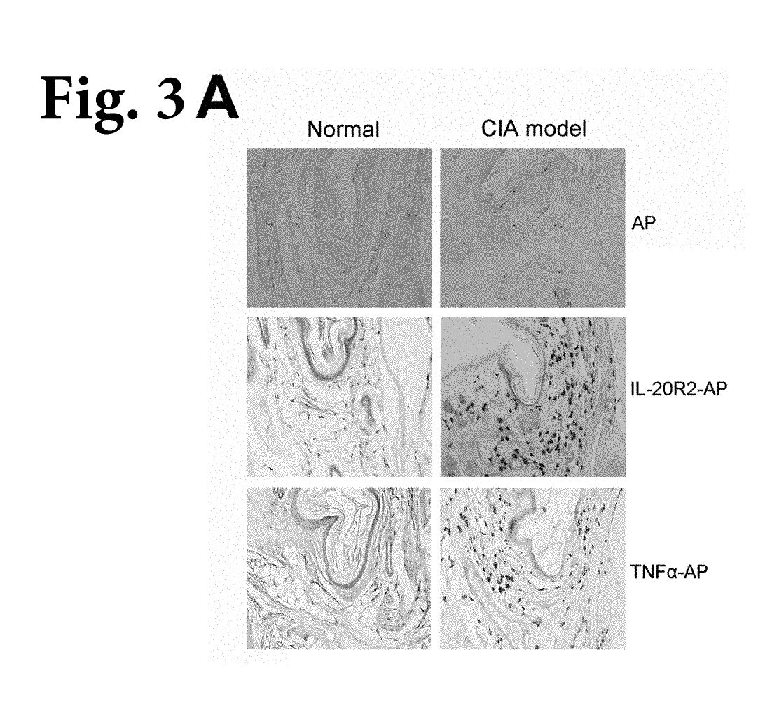

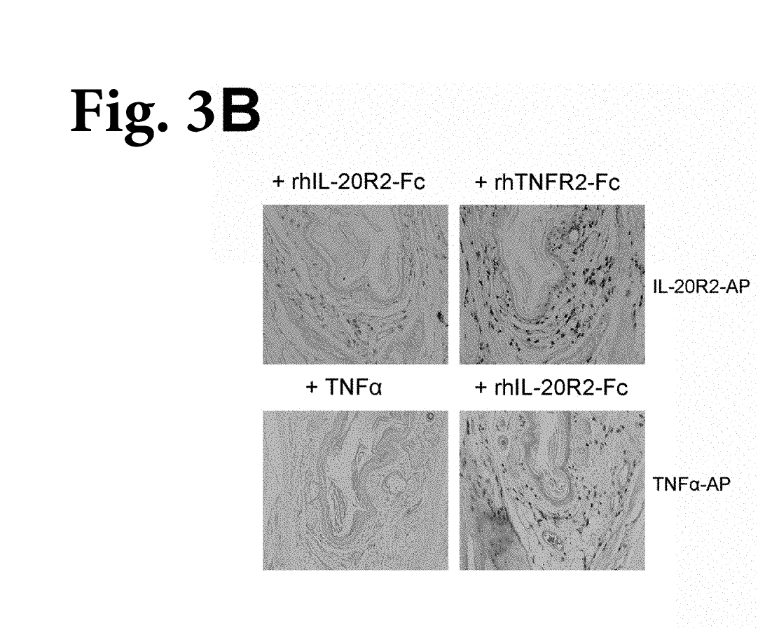

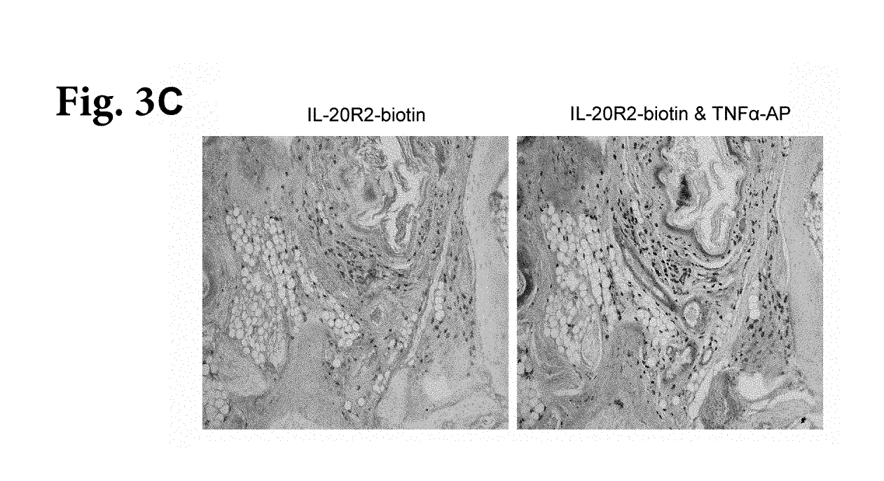

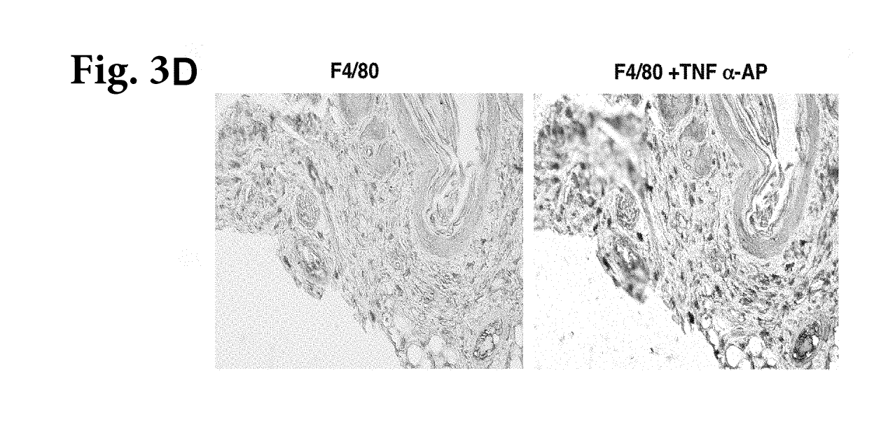

Although abnormal activation in signal transduction pathways via TNFR and IL-20R2 has been both clearly linked to the pathogenesis of RA, the nature of the inter-play and cross-talk between these two signal transduction pathways remain obscure. Past analysis of tissue expression of these cytokines and their receptors in RA from both human patients and animal models have relied on the immunohistochemistry using antibodies that are prone to problems in target specificities. To more accurately evaluate the cell types involved in the production of IL-20 family cytokines and TNFR, paraffin-embedded disease tissues from collagen induced arthritis (CIA) of DBA/1 mice were sectioned and analyzed by in situ ligand-receptor functional staining using AP-tagged soluble IL-20R2 (IL-20R2-AP) and TNF-α-AP. In comparison to normal tissues from control mice that had not been injected with collagen, disease tissues from the swollen paws of CIA mice exhibited a large number of immune infiltrates that were stained strikingly positive for both IL-20R2-AP and TNF-α-AP (FIG. 3A). To ensure that the signals from the in situ ligand-receptor binding assays were ligand-specific, TNFα and rhIL-20R2-Fc were tested for their ability to compete for TNFα-AP or IL-20R2-AP binding, respectively. The results indicated that the positive staining could be largely extinguished by 10-fold excess of the corresponding competitors, supporting that TNFα-AP ligand binding was receptor-specific and IL-20R2-AP receptor binding was ligand-specific (FIG. 3B).

Given the striking similarity in the pattern of both sets of immune infiltrates that populated the disease tissues, we next examined if the expression of TNFR and IL-20 family of cytokines came from the same cell type or neighboring cells. To this end, we conducted a sequential ligand-receptor binding analysis of the CIA tissues by first employing a biotin-labeled IL-20R2 (biotin-IL-20R2) followed by detection of streptavidin-labeled HRP to visualize the expression of the IL-20 sub-family of cytokines. This yielded a pattern of positively stained immune infiltrates (light brown color) similar to that of stained by IL-20R2-AP (FIG. 3C). After capturing the images, the same CIA tissue section was then probed with TNFα-AP followed by visualization of bound AP activity using BCIP substrate which gave darker bluish-purple color. The results revealed that IL-20 family cytokines and TNFα receptor were expressed in the same set of immune infiltrates as the two signals completely overlapped (FIG. 3C). This finding provides a direct evidence that TNF and IL-20R signaling pathways seem to converge in the pathogenesis of rheumatoid arthritis. To identify the cell types that are IL-20R2 and TNF positive in the immune infiltrates, we have conducted a sequential co-staining for anti-F4/80 (macrophage specific) followed by TNFα-AP based on our previous work on IL-24 Transgenic mice. Indeed, we have confirmed that these IL-20R2 and TNF-positive immune infiltrates were macrophages as we suspected, as signals from anti-F4/80 completely overlapped with that of TNFα-AP on the same CIA tissue slide (FIG. 3D).

### Example 4

rhIL-20R2-Fc Ameliorated the Severity of Arthritis in a DBA/1 Mouse CIA Model

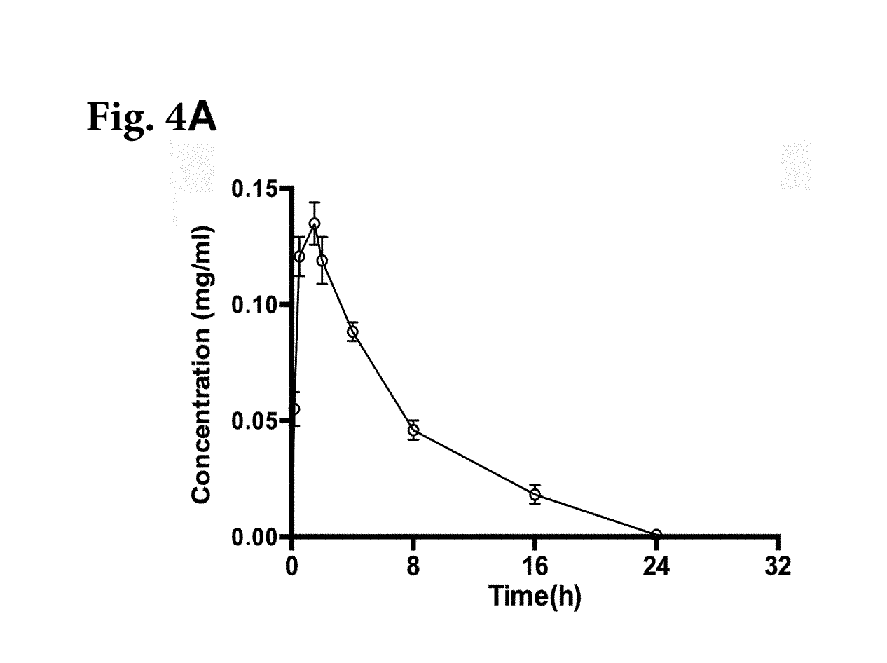

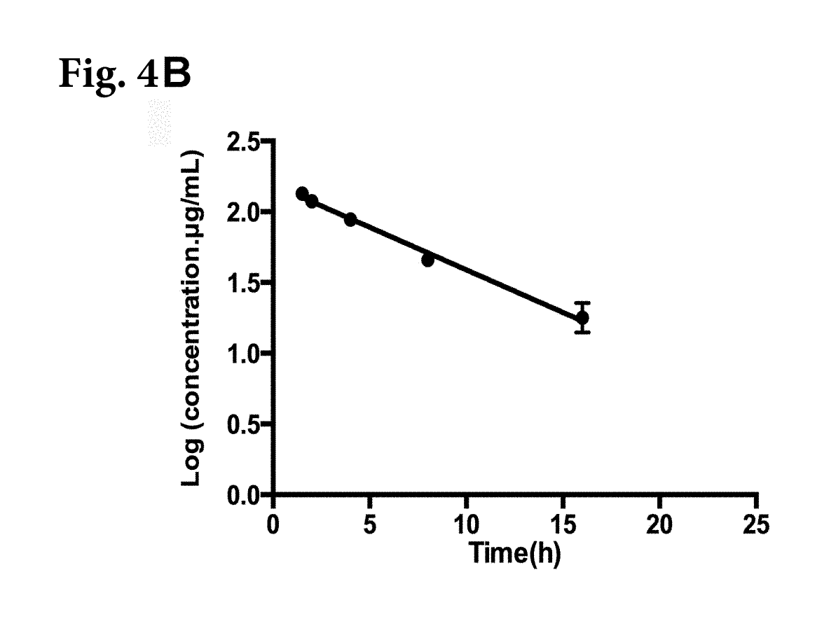

Having demonstrated that rhIL-20R2-Fc can bind to all three ligands (IL-19, IL-20 and IL-24) from IL-20 sub-family and blocks their signaling in vitro, together with the abnormal expression of these cytokines being evident from large numbers of infiltrating leukocytes within disease tissues of RA, we then set out to test if rhIL-20R2-Fc could be therapeutically efficacious in the treatment of collagen-induced arthritis. To determine the dosing strategy, we first conducted pharmacokinetic (PK) study of rhIL-20R2-Fc and showed the half-life of rhIL-20R2-Fc in DBA/1 mouse was approximately 5 hours and AUC₀₋₂₄ₕ=1.033 mg·h/mL (FIG. 4).

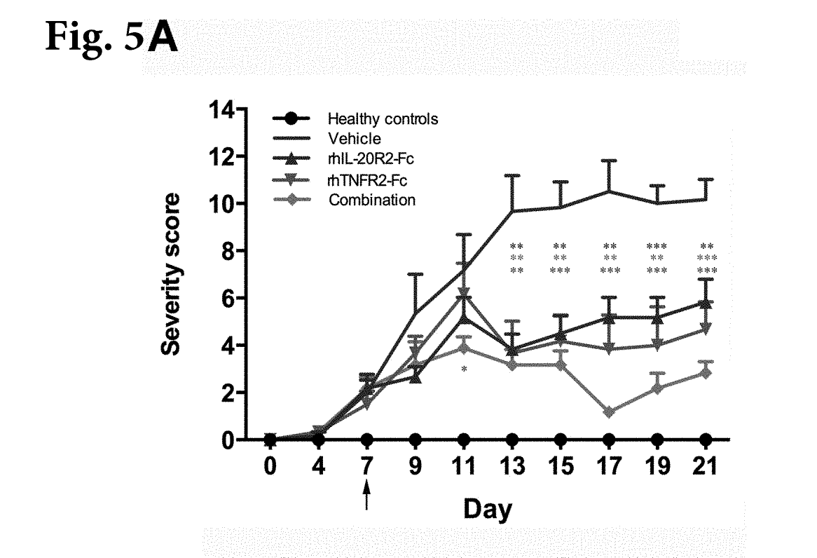

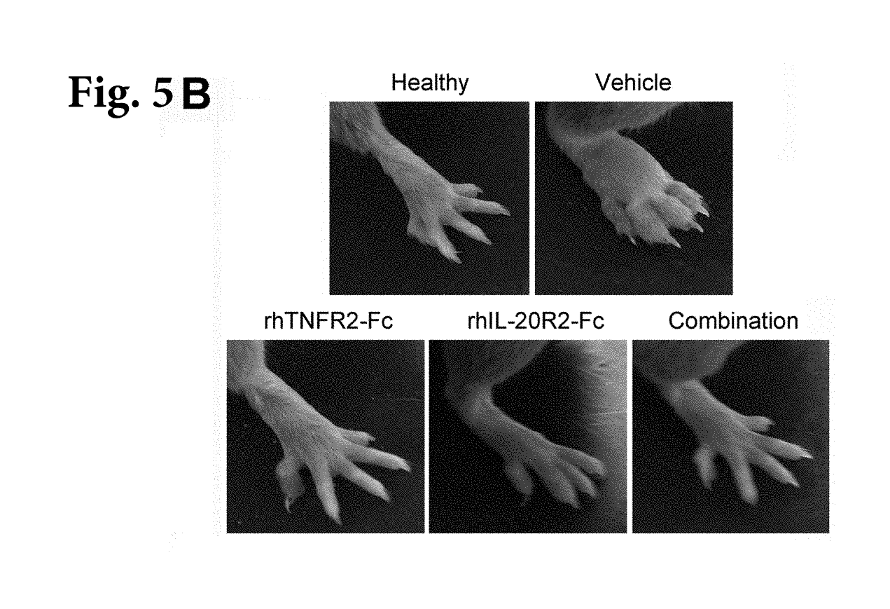

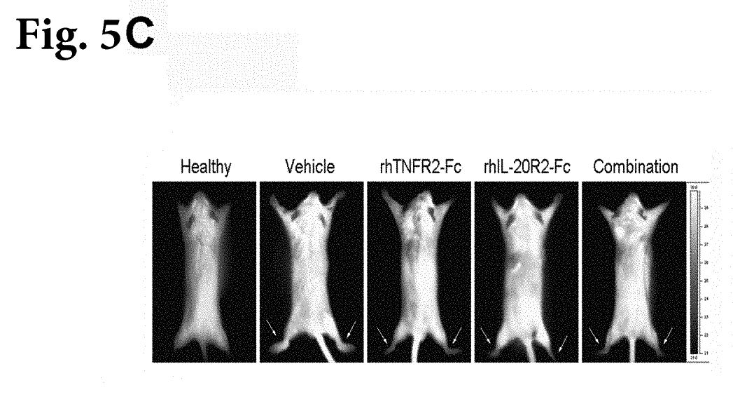

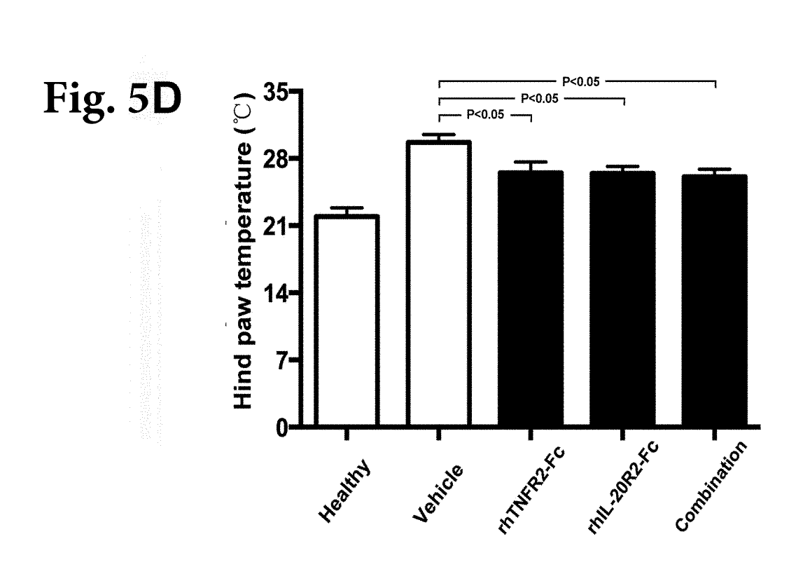

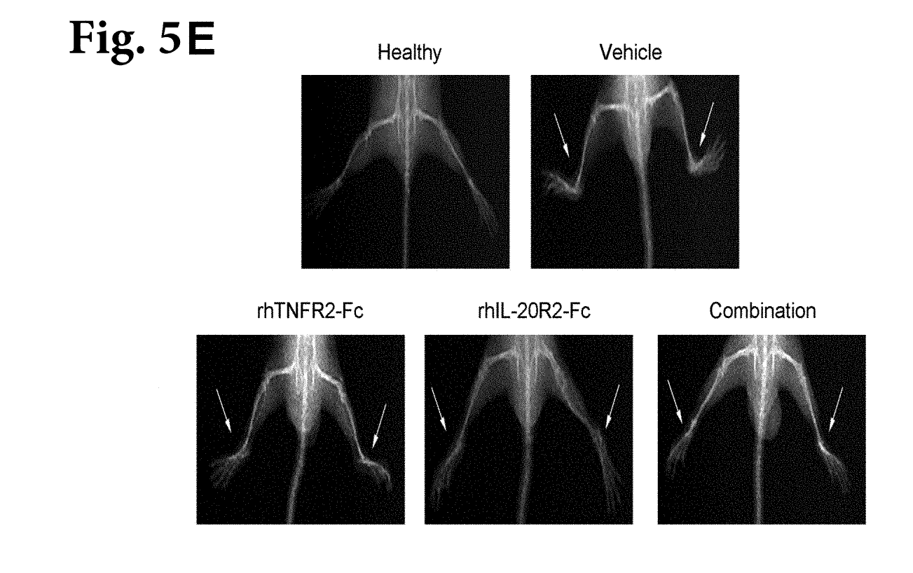

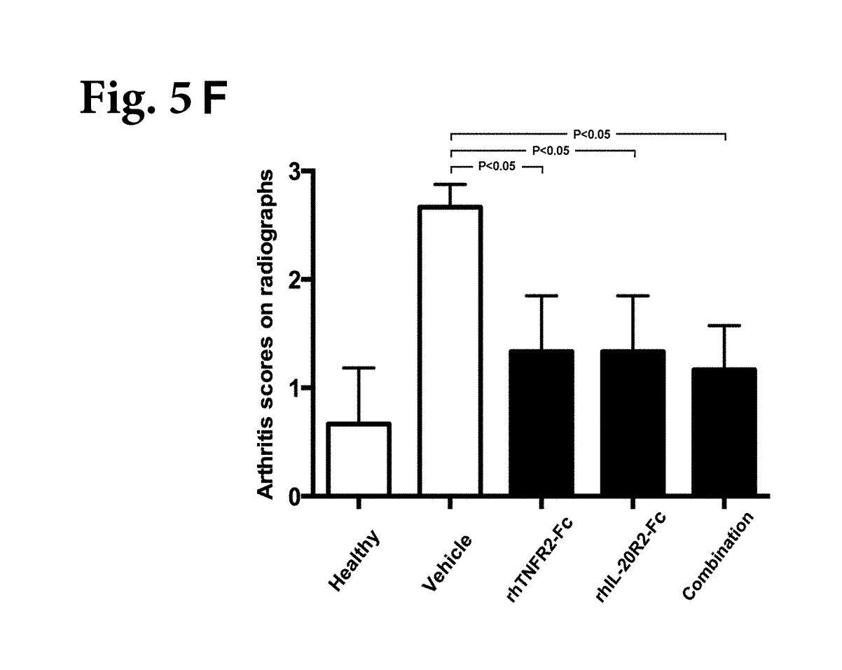

Treatment regimens were followed for the CIA model of DBA/1 mice. Treatment of established RA with rhIL-20R2-Fc injections (i.p) every 16 hours led to significant reduction in the severity scores of RA, compared to non-treatment control (P<0.05) (FIG. 5). As a positive control, the mice were also treated every 48 hours with rhTNFRII-Fc (etanercept) which is the standard first-line biologic treatment for RA in the clinic, as well as a combination therapy with both rhIL-20R2-Fc and rhTNFRII-Fc. The results showed that rhIL-20R2-Fc and rhTNFRII-Fc gave similar efficacy in the treatment of established CIA, each being able to reduce the severity scores of RA by over half compared to non-treated group (FIGS. 5A and B). Moreover, combination therapy resulted in mild synergistic efficacy, although the benefit was not additive. The measurements in RA severity scores were corroborated with both infrared and radiography analysis, which supported the pharmacodynamic functions of rhIL-20R2-Fc in reducing the severity of joints damage for CIA mice (FIGS. 5C, D, E and F).

These important findings provide direct evidence that TNF and IL-20R2 signaling pathways converge on the pathogenesis of RA. It is tempting to hypothesize that the two pathways may function in tandem via macrophages as a relay system in amplifying or maintaining the inflammation where TNF-α acts as a signal input and its receptor activation leads to the production of IL-20 sub-family of cytokines, which in turn may attract more leukocytes to the site of inflammation as shown in IL-24 transgenic mice. Consistent with this hypothesis, rhIL-20R2-Fc, which is able to block all three ligands from signaling through IL-20R2, was as efficacious as etanercept in the treatment of collagen induced arthritis (CIA) in DBA/1 mouse model. Moreover, combinational therapy of etanercept with rhIL-20R2-Fc demonstrated little synergistic effect, supporting that the two pathways operate in tandem, rather than independently in the pathogenesis RA. The therapeutic effects seen are similar to that of individual mAbs targeting IL-19 and IL-20, each of which also exhibited similar efficacy as etanercept. Given that multiple cytokines from the IL-20 family appear to be involved in the pathogenesis of RA in rodent CIA models and that anti-human IL-20 mAb previously yielded inferior therapeutic benefits to that of anti-TNF biologics in human clinical trial for RA, the development of rhIL-20R2-Fc as a broad inhibitor of IL-20 family of cytokines may provide a more effective blockade against signaling from IL-20R2, thus offering a potential new treatment option for RA and/or related diseases. Conceivably, similar goal may also be achievable using therapeutic mAbs against the IL-20R2 receptor subunit, instead of individual ligands of IL-20 family of cytokines.

### Example 5

### Evidence for Autocrine Signaling for IL-20 Family of Cytokines in Human Cancer

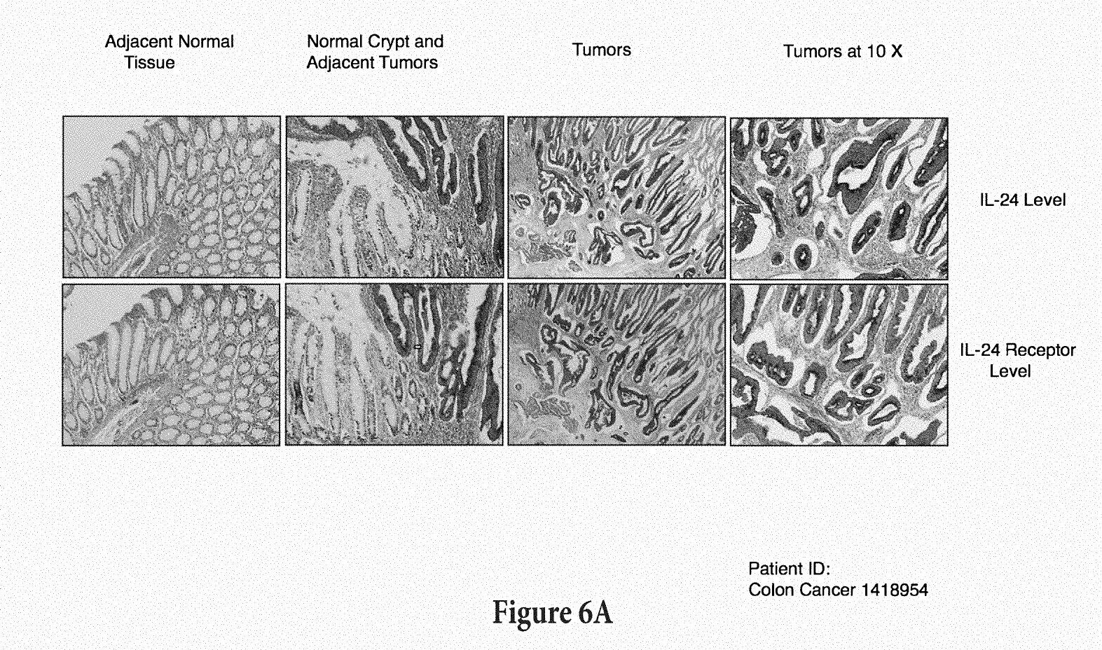

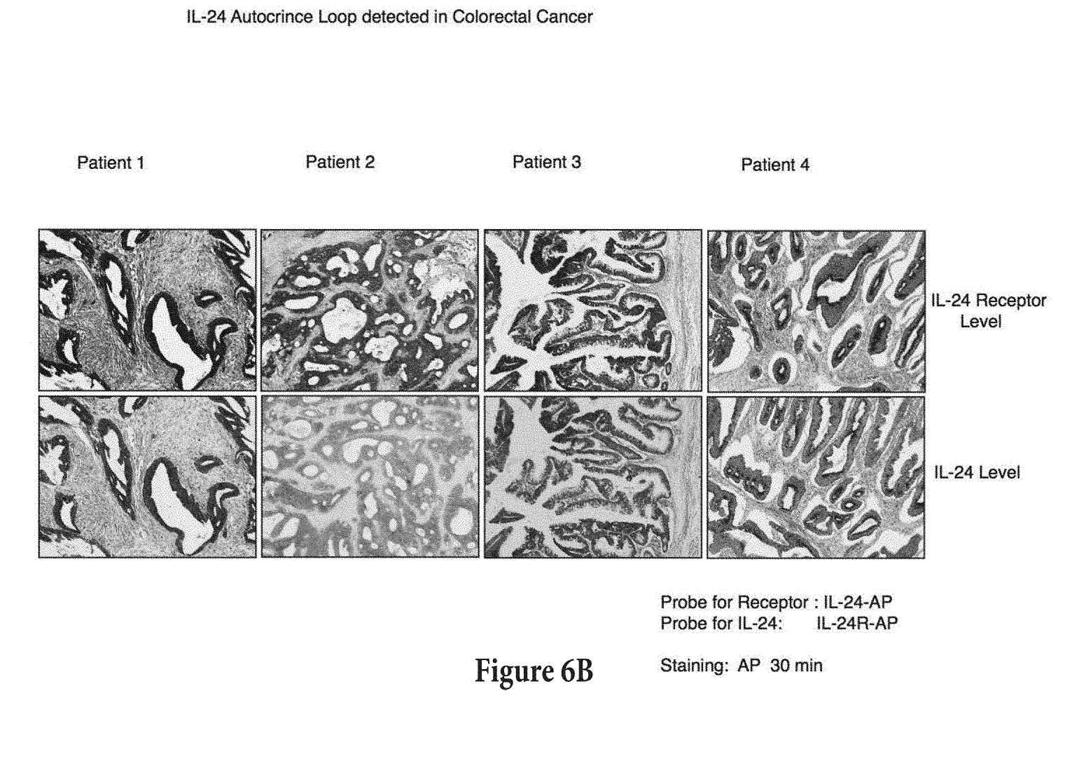

We have discovered IL-24 as a Ras oncogene target, and oncogenic mutations in both K-ras and H-ras lead to over expression of IL-24. We hypothesized that cancer cells may utilize autocrine mechanism to facilitate their abnormal growth and survival by secreting IL-20 family of cytokines which then bind back to their cell surface receptors. To determine if human cancer cells express both IL-20 family of cytokines and their receptors, we used alkaline phosphatase-tagged ligand and soluble IL-20R2 as probes to conduct in situ staining of paraffin-embedded human colorectal cancer tissue specimens. We discovered that cancer cells, but not the adjacent normal tissues, from multiple human colorectal cancer patients were stained strikingly positive for both the ligand and receptor expression using either human IL-24-AP or human soluble IL-20R2-AP as probe (FIGS. 6A and B). This important finding provides a direct evidence and a scientific rationale for blocking IL-20 family of cytokines from activating their receptors in an autocrine fashion. To this end, either rhIL-20R2-Fc which would intercept all three ligands before their binding to their heterodimeric receptors, or antagonist monoclonal antibodies to IL-20R2 receptor subunit, which upon binding to the receptor can prevent all three ligands from IL-20 family from binding and activating their functional receptors on cancer cell surface.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A method for blocking the activity of the IL-20 family of cytokines in a mammal having a disorder-mediated by one or more of the IL-20 family of cytokines comprising the step of administering to said mammal a therapeutically effective amount of a soluble IL-20R2-IgG Fc fusion protein.

2. The method of claim 1, wherein the disorder-mediated by one or more of the IL-20 family of cytokines is an inflammatory disorder.

3. The method of claim 2, wherein the disorder-mediated by one or more of the IL-20 family of cytokines is an arthritis condition.

4. The method of claim 2, wherein the disorder-mediated by one or more of the IL-20 family of cytokines is psoriasis.

5. The method of claim 2, wherein the disorder-mediated by one or more of the IL-20 family of cytokines is atopic dermatitis.

6. The method of claim 2, wherein the disorder-mediated by one or more of the IL-20 family of cytokines is Cohn's disease.

7. The method of claim 1, wherein the disorder-mediated by one or more of the IL-20 family of cytokines is a heart disease.

8. The method of claim 1, wherein the disorder-mediated by one or more of the IL-20 family of cytokines is cancer.

9. The method of claim 1, wherein the disorder-mediated by one or more of the IL-20 family of cytokines is a wound healing disorder.

10. The method of claim 1, wherein the step of administering is performed by intravenous injection.

11. The method of claim 1, wherein the step of administering is performed by subcutaneous injection.

12. The method of claim 1, wherein the step of administering is performed by a series of doses separated by intervals of days or weeks.

13. The method of claim 1, wherein the soluble IL-20R2 in IL-20R2-IgG Fc fusion is a human receptor subunit.

14. The method of claim 1, wherein the IgG Fc in the IL-20R2-IgG Fc fusion protein is human.

15. The method of claim 1, wherein the IgG Fc is human.

16. The method of claim 1, wherein the IgG Fc is from human IgG1.

17. The method of claim 1, wherein the IL-20R2-IgG Fc fusion protein is identified by Sequence ID No. 1.

18. A method for blocking the activity of the IL-20 family of cytokines in a mammal having a disorder-mediated by one or more of the IL-20 family of cytokines comprising the step of administering to said mammal a therapeutically effective amount of a neutralizing monoclonal antibody against IL-20R2, wherein the neutralizing monoclonal antibody, upon binding to IL-20R2, is able to block the IL-19, IL-20 and IL-24 ligands of the IL-20 family from binding to their natural heterodimeric receptors comprising an IL-20R2 subunit, thereby inhibiting signal transduction by IL-19, IL-20, and IL-24.

19. The method of claim 18, wherein the disorder-mediated by one or more of the IL-20 family of cytokines is an inflammatory disorder.

20. The method of claim 18, wherein the disorder-mediated by one or more of the IL-20 family of cytokines is an arthritis condition.

21. The method of claim 18, wherein the disorder-mediated by one or more of the IL-20 family of cytokines is psoriasis.

22. The method of claim 18, wherein the disorder-mediated by one or more of the IL-20 family of cytokines is atopic dermatitis.

23. The method of claim 18, wherein the disorder-mediated by one or more of the IL-20 family of cytokines is Cohn's disease.

24. The method of claim 18, wherein the disorder-mediated by one or more of the IL-20 family of cytokines is a heart disease.

25. The method of claim 18, wherein the disorder-mediated by one or more of the IL-20 family of cytokines is cancer.

26. The method of claim 18, wherein the disorder-mediated by one or more of the IL-20 family of cytokines is a wound healing disorder.

27. The method of claim 18, wherein the step of administering is performed by intravenous injection.

28. The method of claim 18, wherein the step of administering is performed by subcutaneous injection.

29. The method of claim 18, wherein the step of administering is performed by a series of doses separated by intervals of days or weeks.

30. The method of claim 18, wherein the IL-20R2 is a human receptor subunit.

31. The method of claim 18, wherein the neutralizing monoclonal antibody against IL-20R2 is a fully human antibody.

32. The method of claim 18, wherein the neutralizing monoclonal antibody against IL-20R2 is a humanized antibody.

33. The method of claim 18, wherein the neutralizing monoclonal antibody against IL-20R2 is a chimeric antibody.

34. The method of claim 18, wherein the neutralizing monoclonal antibody against IL-20R2 is a Fab.

35. The method of claim 18, wherein the neutralizing monoclonal antibody against IL-20R2 is a single chain antibody.

36. The method of claim 18, wherein the neutralizing monoclonal antibody against IL-20R2 is part of a bi-specific antibody.
